(19) United States
(12) Patent Application Publication
(10) Pub. No.: US 2006/0095098 A1
(43) Pub. Date: May 4, 2006

(54) Hand-held laser device with base station

(76) Inventors: Steven C. Shanks, Mesa, AZ (US); Kevin B. Tucek, Mesa, AZ (US)

Correspondence Address: ETHERTON LAW GROUP, LLC, 5555 E. VAN BUREN STREET, SUITE 100, PHOENIX, AZ, 85008, US

(21) Appl. No.: 10/976,480

(22) Filed: Oct. 29, 2004

(51) Int. Cl. A61N 5/06; A61B 18/18

(52) U.S. Cl. 607/89; 606/13

(57) ABSTRACT

A system for charging and programming one or more hand-held lasers. Each hand-held laser probe contains a rechargeable battery and means for storing program instructions. A base station transmits program instructions to the probe while the probe rests in the base or over the air by using radio or infrared frequencies. The probe may also be programmed by a portable memory unit. The base station is preferably connected to the internet, a telephone network, or computer. In the preferred embodiment, the probe fits into a cavity on the base station and the base station charges the batteries while the laser rests in cavity. The base is connected to house current.

### BACKGROUND

This invention relates generally to medical devices that employ lasers. More particularly, this invention relates to a system for programming and charging a hand-held laser device.

Low energy laser therapy (LLLT) is used in the treatment of a broad range of conditions. LLLT improves wound healing, reduces edema, and relieves pain of various etiologies, including successful application post-operatively to liposuction to reduce inflammation and pain. LLLT is also used with dermatological procedures and during liposuction procedures to facilitate removal of fat by causing intracellular fat to be released into the interstice. It is also used in the treatment and repair of injured muscles and tendons.

Most LLLT devices have a wand containing one or more laser diodes. The wand is attached by a cord to a programming unit, which contains a control circuit that controls the operation of the laser diodes. The cord also conducts electricity to the wand from the programming unit, which is typically battery powered. Usually a practitioner holds the programming unit in one hand and holds the wand in the other hand while treating the patient. The batteries contribute significant weight to the programming unit and on occasion the cord gets tangled or gets in the way of the treatment. It would be desirable to have a lighter device that avoids problems with the cord.

There are a number of variables in laser therapy including the wavelength of the laser beam, size and shape of the area impinged by the laser beam, laser energy, pulse width, treatment duration and tissue characteristics. The success of each therapy depends on the relationship and combination of these variables. For example, liposuction may be facilitated with one regimen utilizing a given wavelength and treatment duration, whereas pain may be treated with a regimen utilizing a different wavelength and treatment duration, and inflammation a third regimen. The practitioner determines the settings of the variables and sets the controls for the programming unit laser accordingly. Some protocols may be stored in the programming unit and recalled later at the time of treatment.

Often it is desirable to treat a patient with multiple types of treatments at the same time. Because specific therapies require different regimens, treating multiple problems currently requires multiple laser devices. Similarly, if the practitioner has several patients being treated at the same time, each patient must wait until the other patient's treatment is completed in order to have access to the laser device. Even if the practitioner has more than one device, he or she must still be present during treatment to maintain control over the laser protocol applied. It would be desirable to have a laser system that can simultaneously provide multiple treatments while ensuring the practitioner maintains control of the protocol(s) used.

Therefore, an object of this invention is to provide a cordless laser system. It is another object to provide a laser system that can simultaneously provide multiple treatments while ensuring the practitioner maintains control of the protocol(s) used.

### SUMMARY OF THE INVENTION

The present invention is a system for charging and programming one or more hand-held lasers. Each hand-held laser probe contains a rechargeable battery and means for storing program instructions. A base station transmits program instructions to the probe either while the probe rests in the base or over the air by using radio or infrared frequencies. The probe may also be programmed by a portable memory unit. The base station is preferably connected to the internet, a telephone network, or computer. In the preferred embodiment, the probe fits into a cavity on the base station and the base station charges the batteries while the laser rests in cavity. The base is connected to house current.

### BRIEF DESCRIPTION OF THE DRAWINGS

FIG. 1 is top view of the base station.

FIG. 2 is a schematic illustration of the base.

FIG. 3 illustrates the probe resting in the base.

FIG. 4a is a rear view of the probe.

FIG. 4b is a front view of the probe.

FIG. 5 is a schematic illustration of the probe.

FIG. 6 is a rear view of an alternate embodiment of the probe illustrating various communication and charging ports.

### DETAILED DESCRIPTION OF THE INVENTION

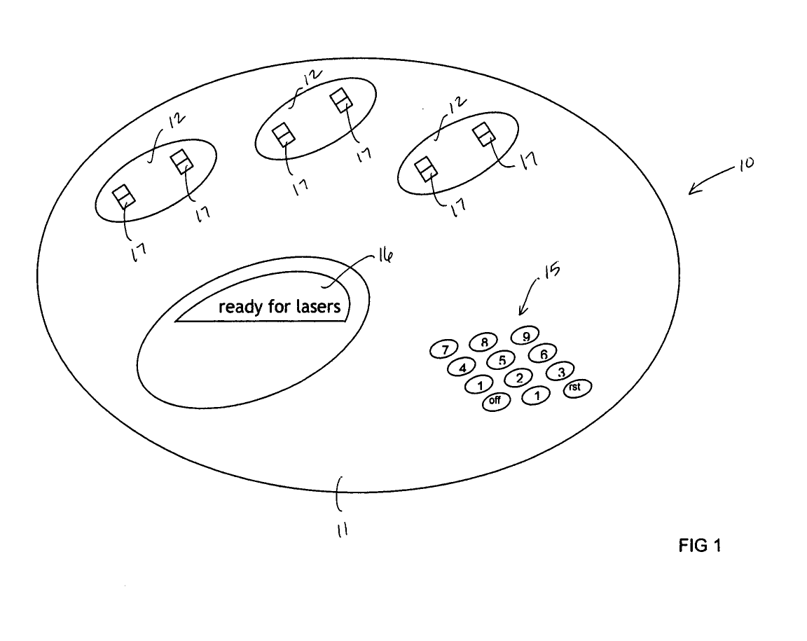

The present invention comprises a base station and a hand-held laser device, referred to herein as a probe 40. The base station can charge the probe, program the probe, or both. FIG. 1 illustrates the base station 10 which comprises a housing 11, at least one cavity 12 for receiving a probe, a keypad 15, and a display 16. Preferably the base station 10 also comprises a battery charger 22 for charging the probe 40. Each cavity 12 in the base station has charging contacts 17 which are accessible in the cavity 12 and which can be contacted with mated contact surfaces 47 of the probe 40. Preferably the base station can accommodate more than one probe.

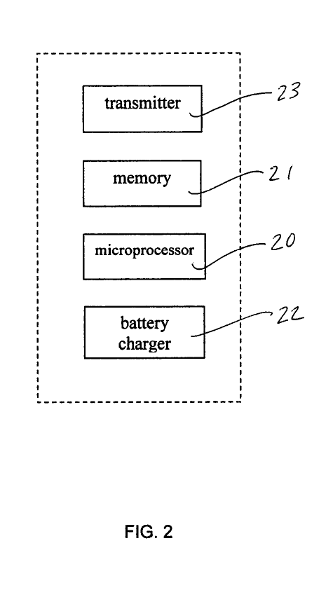

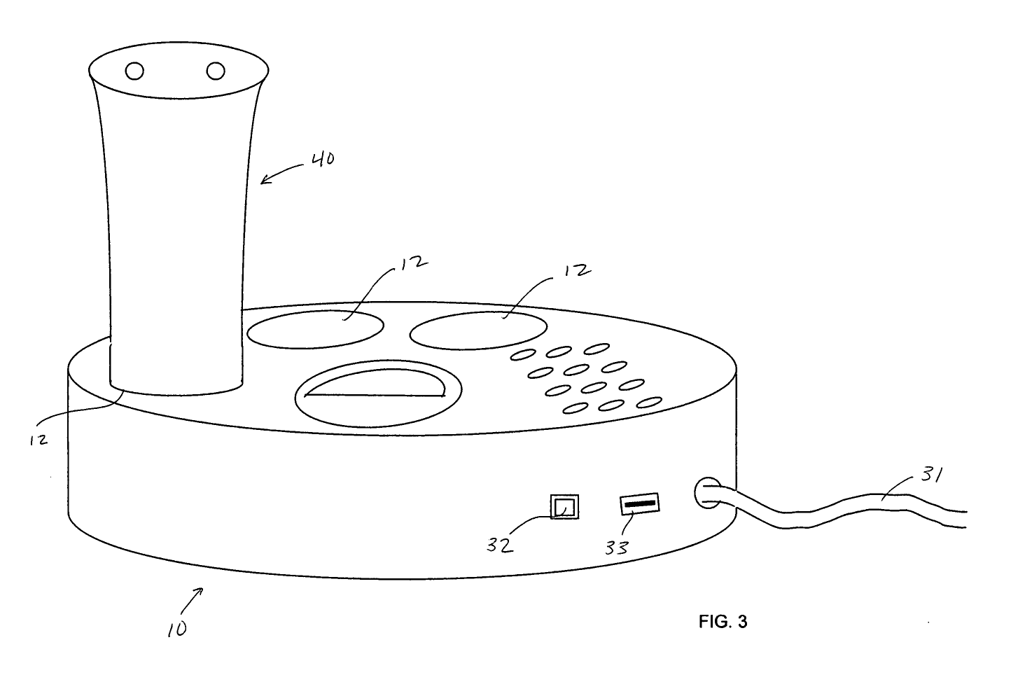

The base station also has means for processing data and means for storing data. The means for processing and means for storing can be implemented in circuitry, either in discrete components or integrated circuits. In the preferred embodiment, the base station 10 contains at least a transmitter 23 for transmitting information to the probe 40, a microprocessor 20 and a memory chip 21. See FIG. 2. The base station 10 is connected to a power source (not shown). The power source is preferably alternating current such as that provided by conventional building current that is then converted to direct current, but may it may also be direct current, such as that provided by a battery. FIG. 3 illustrates a cord 31 to connect the base to mains supply of alternating current.

The base station is programmable and the programming can be accomplished in several ways. The base station may be equipped with a keypad 15 and the user may enter the desired information, such as a treatment program, into the base station using the keypad 15. The base station may alos be equipped with a connector port for receiving one or more memory units containing program information, such as a data disk or memory stick. The base unit may be equipped with one or more connector ports for connecting to data sources such as a telephone network, the Internet, or a computer. FIG. 3 shows the base station with a standard data port 32 and a USB port 33, for receiving information into the base station. Once connected, the desired programs can be downloaded to the base station and then conveyed to the probe, as described in more detail below.

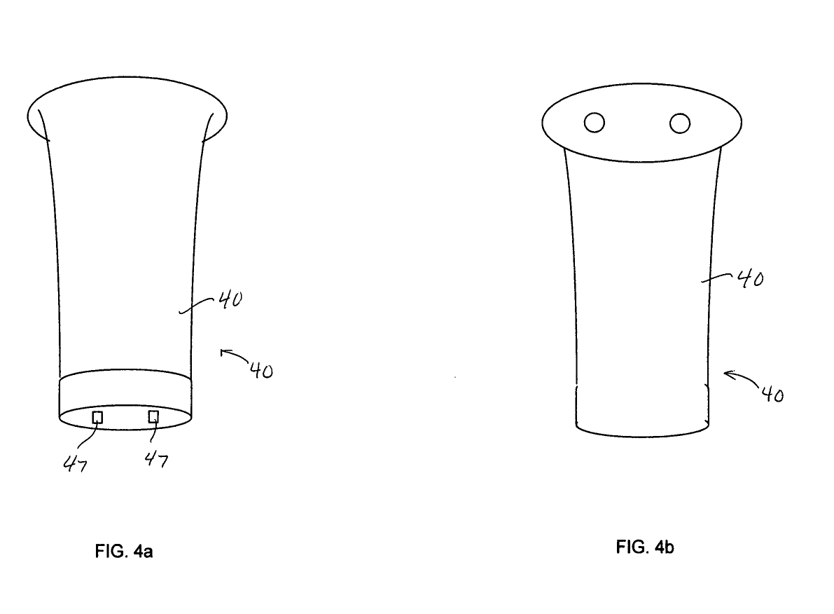

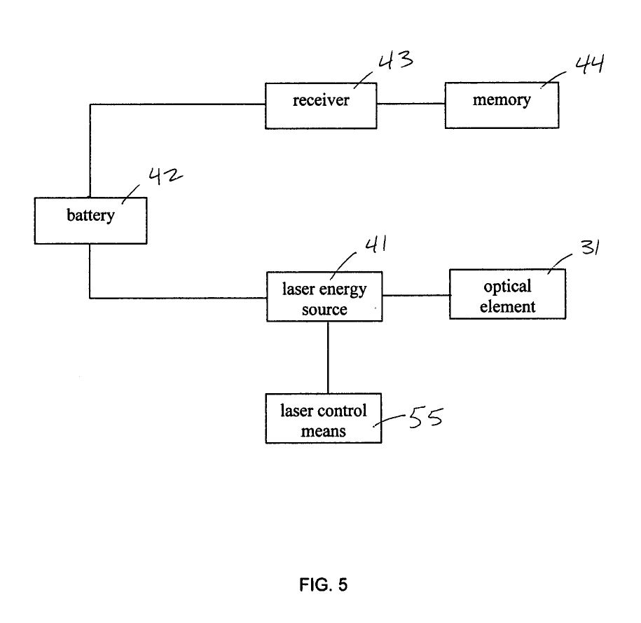

FIGS. 4a and 4b illustrate the probe 40. The probe is sized to be comfortably held in the hand of practitioner. The probe 40 comprises at least one laser energy source 41, a battery 42, a receiver 43, and means for storing data 44. In the preferred embodiment, the battery is rechargeable and the means for storing data in the probe is a flash-programmable memory chip. The probe 40 may also contain one or more switches to turn the probe on and cause laser light to be emitted per the programmed treatment method.

In the preferred embodiment, the probe emits low-level laser energy. Laser energy sources are known in the art for use in low-level laser therapy. They include Helium-Neon lasers having a 632 nm wavelength and semiconductor diode lasers with a broad range of wavelengths between 600-800 nm. The preferred embodiment uses two laser energy sources 41, preferably two semiconductor laser diodes that produce light in the red range of the visible spectrum, having wavelengths of about 635 nm. Other suitable wavelengths are used for other particular applications. While many LLLT regimens include visible laser light, it is advantageous to utilize at least one laser beam in the visible/UV energy spectrum so that the operator can see the laser light as it impinges the patent's body and the area treated can be easily defined. Solid state and tunable semiconductor laser diodes may also be employed to achieve the desired wavelength.

Each laser energy source emits a laser beam through a lens. If a desired beam spot is desired, the laser beam is shone through an optical arrangement 31. The beam spot is the cross-sectional shape and size of the emitted beam as it exits the optical arrangement. For example, a laser beam of circular cross-section creates a circular beam spot as the laser light impinges the patient's skin. If the laser light emitted is in the visible range, a circular spot can be seen on the patient's skin of substantially the same diameter as the laser beam emitted from the optics arrangement. In the preferred embodiment, a first laser beam is passed through an optical arrangement that generates a beam of substantially linear cross-section, resulting in a line of laser light seen on the patient's skin. A second laser passes through an optical arrangement that generates a beam of circular cross-section, resulting in a circular spot shape as seen on the patient's skin.

Control means 55 are connected to the laser energy sources 41 to form a control circuit that control the duration of each pulse of laser light emitted, referred to herein as the pulse width. When there are no pulses, a continuous beam of laser light is generated. Pulse widths of at least one-millionth of a second may be employed to achieve the desired effect on the patient's tissue. The goal of LLLT regimen is to deliver laser energy to the targeted tissue utilizing a pulse width short enough to sufficiently energize the targeted tissue and avoid thermal damage to adjacent tissue. Preferably the laser emission is less than one watt.

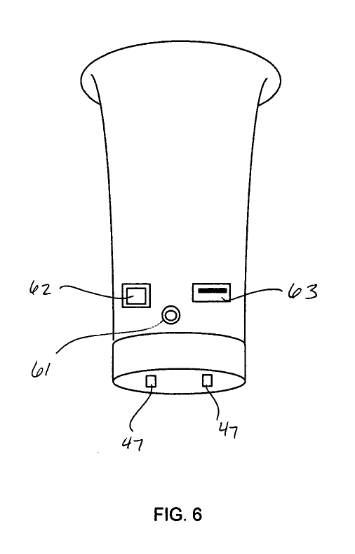

The base station 10 communicates with the probe 40 in several ways to convey program instructions. In the preferred embodiment, the base station 10 is equipped with one or more cavities 12 to receive the probe 40. As explained above, each cavity has charging contacts 17 mated to charging contacts 47 on the probe 40. These contacts may serve not only to charge the probe, but to enable the probe to receive information stored in the base station, or both. Alternatively, the probe may be equipped with a connector port for connecting to the base station by wire. For example, a USB port may be utilized. FIG. 6 illustrates an alternative embodiment of the probe with various communication and charging ports including a charging port 61 for connection to a transformer converting house AC current to direct current; standard data port 62; and a USB port 63. Alternatively, the probe can receive information over the air from the base station using modulatable, electromagnetic waves, such as radio or infrared frequencies, as is known in the art. In such case, the probe 40 contains a receiver 64. The probe 40 has memory means 44 for storing program information. Preferably the laser uses a flash-programmable memory device that would store the received information.

To use the laser system, the base station is connected to a power source, preferably house mains. One or more treatment protocols are programmed into the base station and stored in memory. A hand-held laser probe containing a rechargeable battery is connected to the programmed base station, preferably by seating the probe in a cavity shaped to receive the probe. Connectors in the cavity mate to connectors on the probe and charge the battery in the probe. The probe is programmed with a desired protocol by transmitting a protocol stored in the base station to the probe, preferably by radio frequency. Once charged, the programmed probe is removed from the base station and turned on, causing laser light to be emitted per the program. When the treatment is complete, the probe is returned to the base for a new program and additional charging, if necessary.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

We claim:

1. A hand-held laser system comprising at least one programmable hand-held laser probe in communication with a base station.

2. The system of claim 1 in which the probe comprises:
a) at least one laser energy source for generating at least one laser beam; and
b) an optical arrangement attached to the probe for receiving at least one laser beam and for transforming the laser beam into a desired spot shape.

3. A system according to claim 2 comprising at least two laser energy source wherein at least two of the laser beams are emitted simultaneously.

4. A system according to claim 2 wherein the probe further comprises control means for controlling the laser energy sources.

5. A system according to claim 2 wherein the laser energy source is less than one watt.

6. A system according to claim 2 wherein at least one of the laser energy sources is a semiconductor diode.

7. A system according to claim 2 wherein the laser device generates a laser beam having a wavelength in the visible range.

8. A system according to claim 2 wherein at least one of the spot shapes is substantially linear.

9. A system according to claim 2 further comprising a first laser beam having a first spot shape and a second laser beam having a second spot shape wherein the first spot shape is substantially linear and the second spot shape is not.

10. The system of claim 1 in which the base station further comprises means for programming the probe.

11. The system of claim 10 in which the means for programming the probe comprises:
a) at least one cavity in the base station that is shaped to receive the probe; and
b) connectors in the cavity mated to connectors on the probe the enable the base station to communicate with probe when the probe is inserted into the cavity.

12. The system of claim 10 in which the means for programming the probe comprises:
a) a wire connected to the base station for transmitting information and;
b) at least one connector port in the probe to receive the wire.

13. The system of claim 12 in which the connector port in the probe is a USB port.

14. The system of claim 10 in which the means for programming the probe comprises:
a) a transmitter in the base station that transmits information to the probe using modulatable, electromagnetic waves; and
b) a receiver in the laser to receive information transmitted by the base station.

15. The system of claim 14 in which the electromagnetic waves are radio frequencies.

16. The system of claim 14 in which the electromagnetic waves are infrared frequencies.

17. The system of claim 1 in which the base station further comprises means for receiving program data.

18. The system of claim 17 wherein the means for receiving program data comprises one or more of:
a) a keypad;
b) connection to the internet;
c) connection to a telephone; or
d) connection to a computer.

19. The system of claim 1 in which the base station further comprises means for charging the probe.

20. The system of claim 1 in which:
a) the probe further comprises at least one rechargeable battery;
b) the base station further comprises at least one cavity that is shaped to receive the probe; and
c) the base station further comprises means for charging the battery when the probe is inserted into the cavity.

21. The system of claim 1 in which:
a) the probe further comprises at least one rechargeable battery and means for receiving information transmitted by the base station;
b) the base station further comprises means for charging the rechargeable battery and means for transmitting information to the probe.

22. The system of claim 1 in which the probe further comprises a port for receiving a memory unit and the probe is programmed by inserting the memory unit into the probe.
